(19) United States
(12) Patent Application Publication
(10) Pub. No.: US 2018/0250138 A1
(43) Pub. Date: Sep. 6, 2018

(54) PROSTHETIC HUMERAL HEAD COMPONENT

(71) Applicant: Imperial Innovations Limited, London (GB)

(72) Inventors: Hamidreza Alidousti, London (GB); Jonathan Robert Thomas Jeffers, London (GB); Roger John Hart Emery, London (GB); Andrew Arthur Amis, London (GB)

(21) Appl. No.: 15/744,350

(22) PCT Filed: Jul. 14, 2016
(86) PCT No.: PCT/GB2016/052140

§ 371 Date: Jan. 12, 2018

(30) Foreign Application Priority Data
Jul. 14, 2015 (GB) 1512277.3

(51) Int. Cl. A61F 2/40

(52) U.S. Cl. CPC A61F 2/4014; A61F 2002/30884

(57) ABSTRACT

A prosthetic humeral head component (60) comprising: a convex external head surface (62); an internal cavity (64) formed opposite to the head surface (62); and a plurality of fixation elements (70) protruding from said cavity (64) oppositely to said head surface (62) for location into a peripheral portion (17b) of an epiphyseal plate (17) of the humerus (14) to secure the component (60) to the humerus (14), wherein the component is formed as a single, unitary piece. The fixation elements may comprise pegs (70), fins (1080), or keels (2090), or combinations thereof.

### FIELD OF THE INVENTION

The present invention relates generally to prosthetic humeral head components. In particular, but not exclusively, the invention relates to ways in which to fit such components securely in position on a patient's humeral head.

### BACKGROUND TO THE INVENTION

As is known in the art, disease (such as rheumatoid arthritis) or injury can require either total or partial shoulder joint surgery, in which a patient's humeral head and, optionally, their glenoid bone surface are replaced by respective prosthetic humeral head and glenoid components. The present invention is primarily concerned with prosthetic humeral head components, but the teachings can also be applied to prosthetic components for replacing the heads of other long bones.

There currently are two main types of humeral head component in use:
humeral head resurfacing components, shown schematically in FIG. 1a (such as the well-known Copeland™ implant); and
short-stemmed humeral head replacement components, shown schematically in FIG. 1b (such as the Affinis™ Short stem, from Mathys European Orthopaedics™)

With a humeral head resurfacing component 10, a thin layer of bone is removed from the end of the humeral head 14 and the implant 10 is secured by a central stem 12 that is received in a corresponding central bone tunnel 16. This often leaves an empty space 13 beneath the implant 10 where bone is excessively eroded due to the disease or injury which necessitated the procedure. As such, this type of implant is not reliably capable of filling up the eroded areas in the humeral head 14. In addition, resurfacing implants are prone to misalignment due to overstuffing (where insufficient bone material is removed).

With a short-stemmed component 20, the entire end of the humeral head 14′ is cut off and the implant 20 is secured by a central stem 22 that is received in a corresponding central bone tunnel 16′. Whereas this ensures a snug interface with no voids between an underside surface 23 of the implant 20 and the opposing resected surface 15 of the humeral head 14′, this also removes all the best bone for fixation, which is that located above the epiphyseal plate or line 17 (see FIG. 3). As a result, this creates a less stable bed for the implant 20 to seat on.

A variant of the humeral head resurfacing component is described in US 2012/0296436A1, and as illustrated in the accompanying FIG. 1c. Here, the component 30 has a cruciform stem 32, having fins 34 extending out radially from a central core 36 at 90° intervals. The fins 34 help to provide stability of the component 30 within the associated bone tunnel 16.

With all of the above-described known components 10, 20, 30, implantation relies upon a relatively large and long central fixation feature that is received in the relatively soft central bone of the humeral head. Such relatively large stems, received in relatively soft bone, can result in deleterious stress-shielding, as well known in the art.

Moreover, the relatively large height of these known components—i.e. the distance from the top 40 of the hemi-spherical external bearing surface 41 to the tip 42 of the stem 12, 22, 32—means that they can only be inserted into position anteriorly during implantation.

These problems are addressed by the various aspects of the current invention.

An object of the invention is therefore to provide a more secure attachment of a prosthetic humeral component to the underlying bone surface.

Another object of the invention is to enable surgeons to insert a prosthetic humeral component posteriorly during a procedure.

Where a prosthetic humeral component becomes loose, it needs to be replaced through revision surgery. Such revision surgery is typically difficult because of the damage caused to the humeral bone by the loosening humeral component and the fact that a larger fixation element is needed to make a secure connection. This can lead to a sink-hole effect during revision surgery, whereby the weak central portion of the humeral bone is destroyed as the surgeon tries to remove the primary component (typically secured in place with bone cement in the initial procedure, or having been subject to bone ingrowth) and bone attached to it comes out, resulting in loss of structural viability for reliably securing the revision component.

Another object of the invention is therefore to provide a prosthetic humeral component that allows for better revision procedures.

### SUMMARY OF THE INVENTION

According to a first aspect of the invention, defined by the accompanying claim 1, there is provided a prosthetic humeral head component comprising:
a convex external head surface;
an internal cavity formed opposite to the head surface; and
a plurality of fixation elements protruding from said cavity oppositely to said head surface for location into a peripheral portion of an epiphyseal plate of a humerus to secure the component to the humerus, wherein the component is formed as a single, unitary piece.

The component may be rotationally symmetrical about an axis. In certain embodiments, the fixation elements are arranged asymmetrically, but the remainder of the component is symmetrical. The internal cavity may be substantially frusto-conical, defined by a chamfered circumferential wall and a ceiling. The circumferential wall may be formed at an angle in the range of 10° to 80° from said axis. In some embodiments, the ceiling is substantially perpendicular to said axis. The ceiling may be planar or may be either concave or convex. A convex ceiling may be advantageous in providing improved load transfer to the surrounding bone.

Preferably, each of the plurality of fixation elements is positioned such that its centre is at a radial distance from a central axis of the component through the convex external head surface that is at least 70% of the radius of the component from the central axis. Preferably, each of the plurality of fixation elements of the component is positioned such that its centre is at a radial distance of at least 15 mm from a central axis of the component through the convex external head surface.

These features ensure that the fixation elements are positioned so as to be located, when fitted to the bone, in the parts of the peripheral region of the bone that are typically the most dense and therefore provide a more secure anchoring.

Where the component has a circumferential wall, that circumferential wall may have a concave surface. The concave surface of the circumferential wall may be substantially parallel to the convex external head surface, thereby defining a skirt of substantially equal thickness depending from the periphery of the ceiling.

In certain embodiments, the internal cavity is vaulted, defined by a chamfered circumferential wall with a concave surface that meets at the axis. In certain other embodiments, the circumferential wall may have a convex surface.

The fixation elements preferably extend from the circumferential wall.

In some embodiments, at least some of the fixation elements protrude beyond a plane defined by an edge of the external head surface.

According to one aspect of the invention, the fixation elements comprise pegs.

The pegs may extend parallel to one another. The pegs may be arranged equidistantly. In certain embodiments, there are four such pegs, although both greater and fewer such pegs are viable alternatives.

Each peg typically comprises a substantially cylindrical body portion and a wider base portion where the peg protrudes from the cavity. Each peg also typically has a rounded free end.

According to another aspect of the invention, the fixation elements comprise fins. Each fin typically comprises a first edge extending radially inwards from a point on the circumferential wall and a second edge extending from a point on the ceiling to a point meeting the first edge. The first edge may extend substantially perpendicularly to the axis, or may be angled either towards or away from the ceiling.

In certain embodiments, one or both of the first and second edges is curved.

The second edge may extend at a more acute angle to the axis than the circumferential wall.

The first and second edges may each have a rounded profile.

The junction between the first and second edges may be rounded.

The first and second edges may in some embodiments be contiguous with one another.

The fins are typically arranged equidistantly from one another, although alternative embodiments in which they are not are also envisaged.

In certain embodiments, there are four such fins, although both greater and fewer such fins are viable alternatives.

According to a further aspect of the invention, the fixation elements comprise keels, each keel extending across the whole of the internal cavity. Each keel may comprise a flat portion aligned parallel to and intersecting the axis, the flat portion having a free edge opposite to the ceiling. The free edge may have a rounded profile.

In some embodiments, a pair of such keels is provided, intersecting at right angles to one another.

The component according to any of the above-described aspects may further comprise additional fixation elements protruding from the ceiling. In certain embodiments, the component may comprise a mixture of different kinds of fixation elements.

The component is typically formed from a single piece of ceramic material, although alternative options are also described for example a metallic alloy, such as cobalt-chrome or titanium.

The component may further comprise an osseo-conductive coating on at least one of the internal cavity and the plurality of fixation elements.

The arrangement facilitates a chamfer cut to be made on the fixation surface—i.e. the surface of the long bone head that is the subject of the procedure—and for secure attachment of the component to the bone surface to be effected by virtue of the peripheral fixation elements (only). It is surgically much easier and less time consuming to prepare a bone with a chamfer cut than cutting off the entire end of the humeral head, as with conventional humeral head components. Additionally, when a chamfer cut is made, the central region of the bone surface remains intact for use in any future revision surgery that might be required, so it is possible to revise to a short stem device rather than a long stem implant. Also, fixation elements are only inserted into the strongest, densest subchondral bone; the provision of multiple fixation elements at the periphery meaning that the overall height of the component can be kept to a minimum, in turn leading to improved surgical access. As opposed to resurfacing procedures, a chamfer cut gives a greater access to the glenoid for a total shoulder replacement.

The shape of the internal cavity means that a thick shell of up to 10 mm can be provided above it. This thick shell is able to fill the eroded regions of the humeral head while the chamfer shape allows the implant to utilise the good quality bone above the epiphyseal plate and the anatomical neck for fixation. The chamfered shape also encases the bone and creates extra stability.

As well as being a monoblock, the component has a shell which is thicker than resurfacing implants and thinner than short stem designs.

### BRIEF DESCRIPTION OF DRAWINGS

Embodiments of the present invention will now be described, by way of example only, with reference to the accompanying drawings, in which:

FIG. 1 a shows a schematic cross-section of a known humeral head resurfacing component;

FIG. 1b shows a schematic cross-section of a known short-stem humeral head component;

FIG. 1c is a perspective view from below of a known humeral head resurfacing component, with a cruciform central stem;

FIG. 2 is a schematic cross-sectional view of a humeral head component according to embodiments of the invention;

FIG. 3 shows sectional images of a humeral head, illustrating the bone structure;

FIG. 4a is a perspective underside view of a prosthetic humeral head component according to one embodiment of the invention, having peripheral pegs as fixation elements; and FIG. 4b is a corresponding plan view from below;

FIG. 5a corresponds to FIG. 4a, but with the fixation elements removed for clarity;

FIG. 5b is a cross-sectional view of the component of FIG. 5a;

FIG. 6a is a cross-sectional view of a variant of the invention, having a shallower hollow cavity than the embodiment of FIG. 5; and FIG. 6b corresponds, but with the fixation elements removed for clarity;

FIG. 7 shows another variant of the invention, having a bulbous, convex circumferential side wall, again with fixation elements removed for clarity;

FIG. 8 depicts an embodiment in which the circumferential wall has a constant thickness, again with fixation elements removed for clarity;

FIG. 9a is a cross-sectional view illustrating another variant of the invention, in which a ceiling of the hollow cavity is convex; and FIG. 9b corresponds, but with the fixation pegs removed for clarity;

FIG. 10 illustrates an embodiment having a convex ceiling and a convex circumferential wall, fixation elements removed for clarity;

FIG. 11 is a cross-sectional view illustrating yet another variant of the invention, in which the internal cavity is vaulted, defined by a chamfered circumferential wall with a concave surface that meets at a central axis, fixation elements removed for clarity;

FIG. 12 is a perspective underside view of a prosthetic humeral head component according to another embodiment of the invention, having peripheral fins as fixation elements;

FIG. 13 is a perspective underside view of a prosthetic humeral head component according to yet another embodiment of the invention, having keels as fixation elements;

FIG. 14 is a perspective underside view of an embodiment having an additional short fixation peg depending from the ceiling;

FIG. 15a is a perspective underside view of an embodiment having multiple additional short fixation pegs depending from the ceiling; FIG. 15b is a plan view from below of the embodiment of FIG. 15a;

FIG. 16a is a perspective underside view of an embodiment having small keels as additional fixation elements depending from the ceiling; FIG. 16b is a plan view from below of the embodiment of FIG. 16a;

FIG. 17 is a perspective underside view of an alternative embodiment having an additional fixation peg depending from the ceiling;

FIGS. 18a-18d depict variant arrangements for embodiments having fins as fixation elements;

FIGS. 19 and 20 depict a range of different sized components overlaid on one another to illustrate their differing external profiles and their common internal profile;

FIG. 21a depicts the variation of humeral bone density from the proximal to distal region across computed tomography (CT) bone slices parallel to the anatomical neck; and

FIG. 21b depicts the variation of humeral bone density from central to peripheral zones for CT bone slices parallel to the anatomical neck.

### DETAILED DESCRIPTION

The following description will be made in the context of a prosthetic humeral component, for use in surgical reconstruction of a shoulder joint to provide a prosthetic humeral head for articulation with a corresponding glenoid surface—either original bone or itself a prosthetic component. It should be understood, however, that the principles and teachings can be applied mutatis mutandis to produce prosthetic components useable to replace articular heads of other long bones, such as the femur.

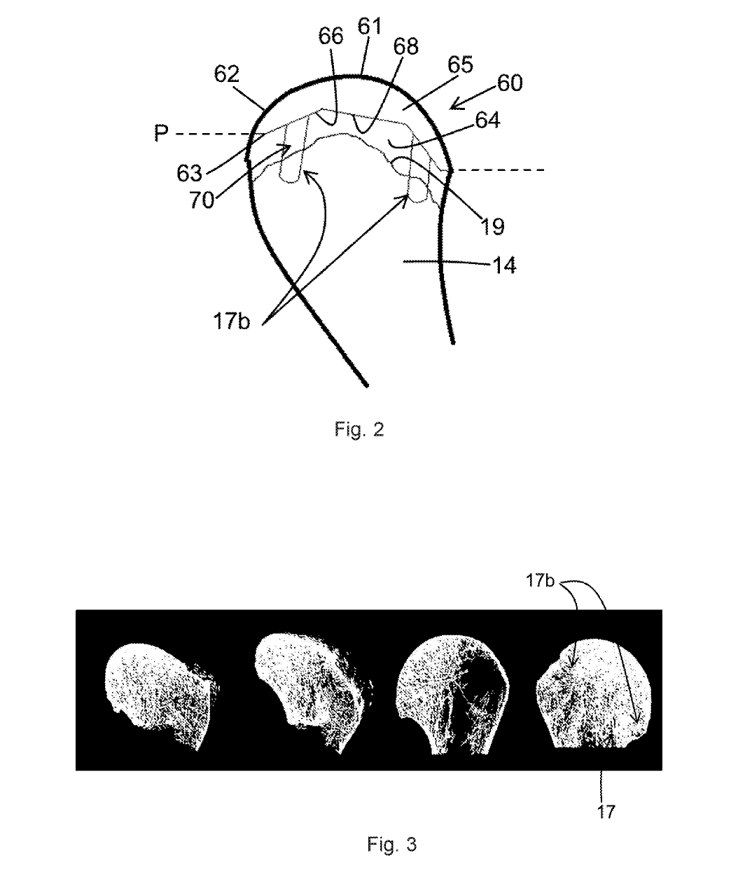

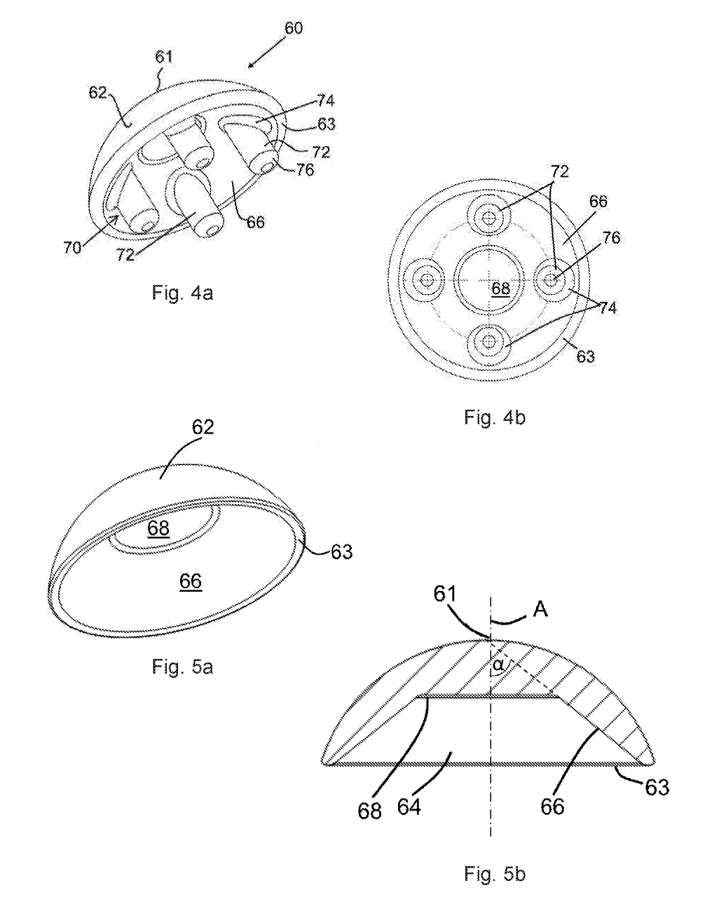

A prosthetic humeral component 60 according to a first embodiment is depicted in FIGS. 2, 4a and 4b, with additional reference to FIGS. 5a and 5b. The component 60 has a conventional convex external head surface 62, which for convenience can be described as hemi-spherical, although in fact the surface may not describe an entire hemi-sphere or may extend further beyond a mid-plane of a spherical surface. The external head surface 62 terminates at an annular edge or rim 63. An internal cavity 64 is formed opposite to the head surface 62. The cavity 64 has a frusto-conical shape and is defined by a chamfered circumferential wall 66 and a ceiling 68. The lower extent of the cavity 64 is defined by a plane P aligned with the rim 63. A relatively thick shell portion 65 of up to 10 mm is formed between the ceiling 68 and the external bearing surface 62. In a preferred embodiment, the depth of the shell portion 65 (i.e. the distance along the axis A from the apex of the external head surface 62 to the ceiling 68) is 45% of the height of the component head (i.e. the distance along the axis A from the apex of the external head surface 62 to the plane P).

The component 60 is rotationally symmetrical about an axis A.

Four pegs 70 are arranged equidistantly at 90° intervals about the axis A and protrude parallel to one another from the cavity 64 oppositely to the head surface 62. Each peg 70 comprises a substantially cylindrical body portion 72 with a diameter, in preferred embodiments, of 7 mm, and a wider base portion 74, where the peg 70 is connected to the circumferential wall 66. Each peg has a rounded free end 76. The component 60, comprising the external convex bearing surface 62, the internal cavity 64 and the pegs 70, is formed as a single, unitary piece, as described in greater detail below.

Because the pegs are arranged in this manner, depending from the circumferential wall 66 rather than centrally from the ceiling 68, when the component is secured to a patient's humeral bone 14 during a surgical procedure the pegs 70 will locate into a peripheral portion 17b of an epiphyseal plate or line 17 of said bone. This will ensure a secure attachment of the component 60 to the bone 14.

Although the pegs 70 of this embodiment are arranged equidistantly and symmetrically about the axis A, it will be understood that in certain embodiments it might be advantageous to have the pegs 70 arranged, peripherally, in a non-symmetrical manner, for example to ensure that they are received in the best possible regions of bone stock. Those best regions of bone stock could be ascertained on a patient-specific basis by reference, for example, to pre-operative imaging data. Additionally or alternatively, the pegs may be positioned so as to be located, when fitted, in the parts of the peripheral region that are typically the most dense, such as in the humeral calcar region. In a preferred embodiment, the pegs 70 are positioned such that their centres are at a radial distance of 15 mm from the axis A.

The inventors carried out a study to investigate the bone density in the proximal areas of the humerus to provide information for implant design and guidance on the most appropriate positions to place implant fixation features, for example pegs 70 in the first embodiment. The study identified that the densest regions of bone are located above the humeral anatomical neck and epiphyseal plate, and also below the anatomical neck at the periphery. As such, one or more of the pegs 70 of the humeral component 60 are located so as to engage with one of these regions.

As part of the study computed tomography (CT) scans of healthy humeri were used to map bone density distribution in the humeral head. The proximal humeral head was divided into twelve slices parallel to the humeral anatomical neck starting from the most proximal region of the humeral head to distal regions beneath the epiphyseal plate. Each slice was then divided into four concentric circles. The slices below the anatomical neck were further divided into radial sectors. The average bone density for each of these regions was calculated and regions of interest were compared using a repeated measures analysis of variance (ANOVA) technique with significance set at p<0.05.

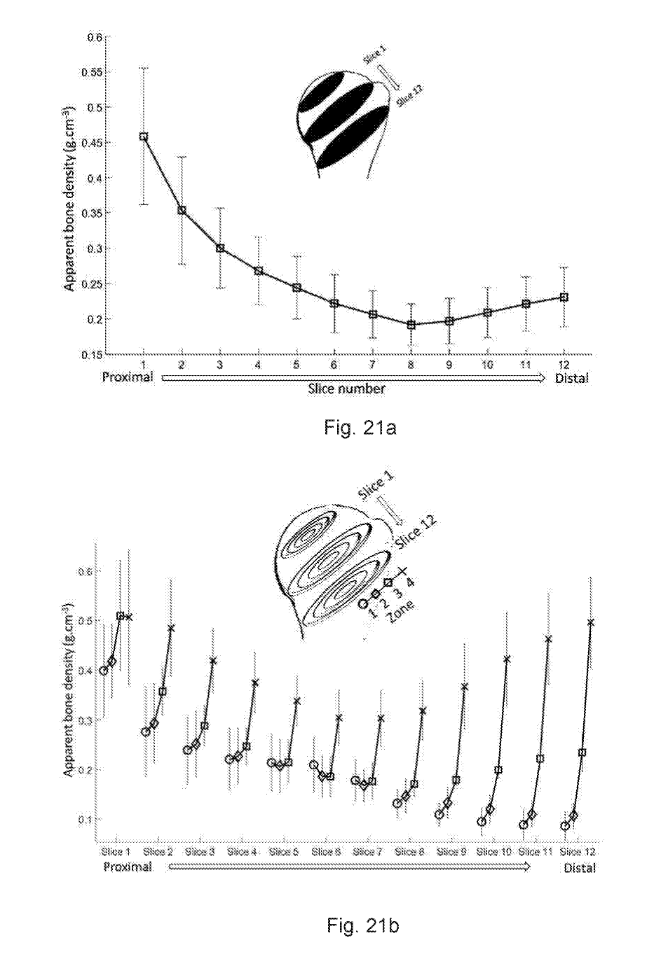

FIG. 21a depicts variation of humeral bone density from the proximal to distal region across computed tomography (CT) bone slices parallel to the anatomical neck. The range and the orientation of the slices are shown, with the anatomical neck at slice 6. Average apparent bone density was found to decrease from proximal to distal regions leaving the majority of higher bone density proximal to the anatomical neck of the humerus (p<0.05).

FIG. 21b depicts the variation of humeral bone density from central to peripheral zones for CT bone slices parallel to the anatomical neck. The range and the orientation of the slices are shown. Below the anatomical neck, bone density increases from central to peripheral regions where cortical bone eventually occupies the space (p<0.05). In distal slices below the anatomical neck, a higher bone density distribution in medial calcar regions was also observed.

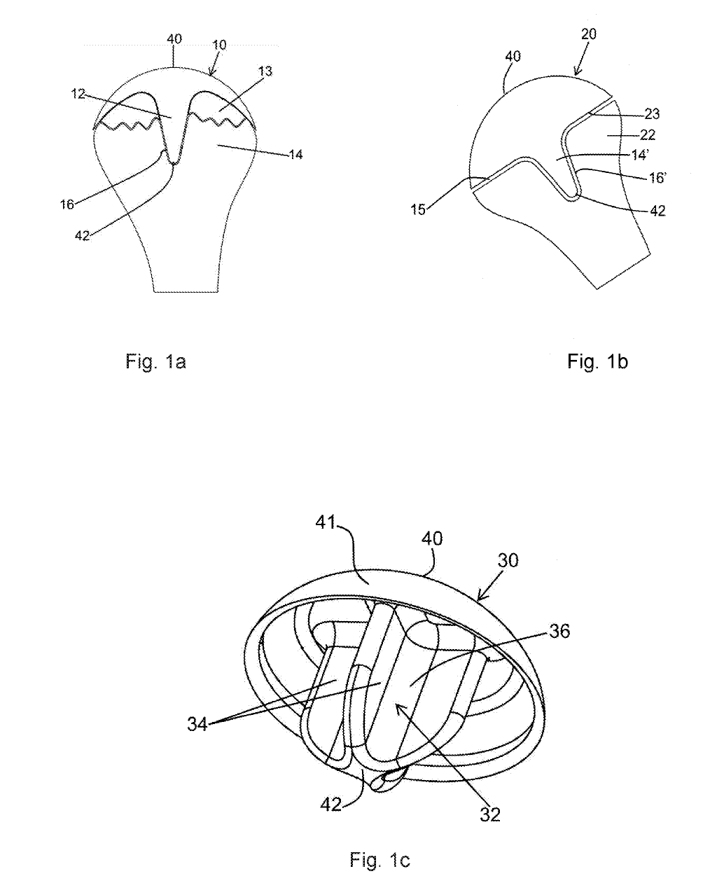

Conventional short stem implants require the humeral head to be resected at the anatomical neck and use central fixation features to fix the implant in the distal bone. The inventors discovered that it is advantageous to preserve bone above the anatomical neck and epiphyseal plate as it exhibits greater density and, therefore, provides improved fixation. Additionally, below the anatomical neck, denser and thus stronger bone is located at the periphery which is in contrast with the design philosophy of conventional short stem designs that employ central fixation, such as those depicted in FIGS. 1a, 1b and 1c.

Using the results of the study, the inventors designed the various embodiments of the present humeral head component so as to provide improved fixation over conventional humeral head components based on the fact that they leave more of the denser bone in the proximal region in place and also because the fixation elements are located for insertion into a peripheral portion of an epiphyseal plate of a humerus.

In the pre-operative planning stage, imaging data of the relevant anatomy of a patient can be obtained using one of the medical imaging methods described above. The imaging data obtained and other associated information can be used to construct a three-dimensional computer image of the relevant portion of the anatomy of the patient. An initial pre-operative plan can be prepared for the patient in image space and can include bone or joint preparation, planning for resections, milling, reaming, broaching, implant selection and fitting, as well as designing patient-specific guides, templates, tools and alignment methods for the surgical procedure.

Moreover, the provision of four pegs 70 is just one exemplary embodiment, and it will be understood that greater or fewer pegs 70 may be provided. The number of pegs, and their particular arrangement, may be determined on a patient-specific basis.

In certain embodiments, the pegs 70 may project from the cavity 64 at an angle to the axis A rather than being parallel to it.

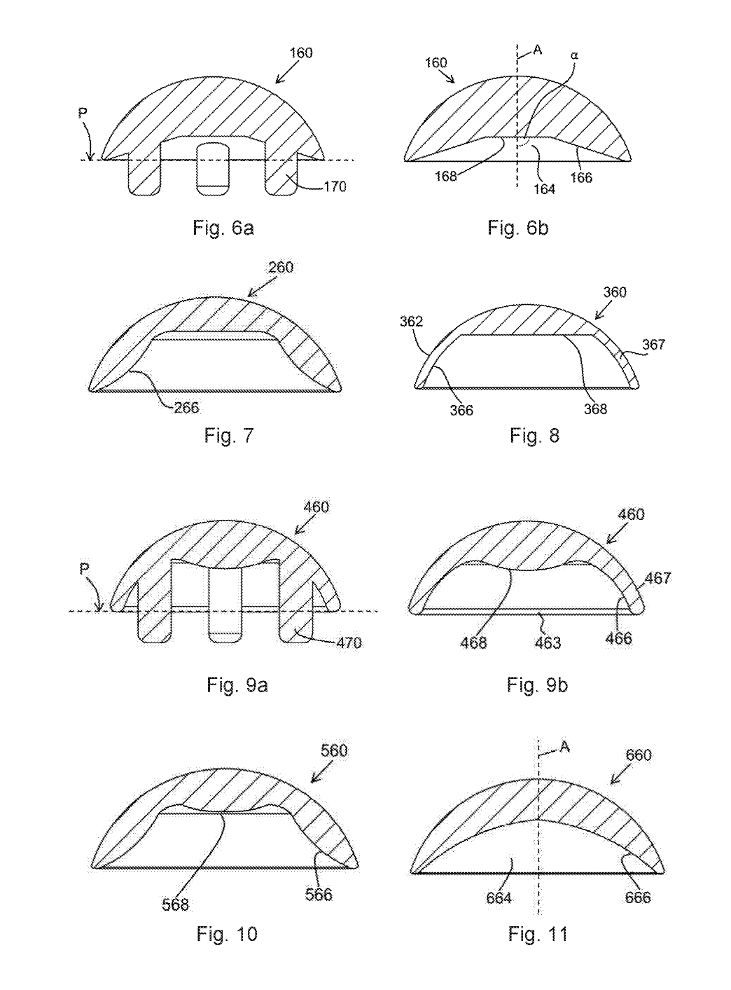

In the embodiment of FIGS. 4a-5b, the circumferential wall 66 is at an angle α of approximately 45° relative to the axis A. In an alternative embodiment, of FIGS. 6a and 6b, the angle α is less acute, being about 70°, resulting in a shallower cavity 164 and a thicker shell 165. A more acute angle would result in a deeper cavity 64, 164 but a thinner shell. The circumferential wall 66, 166 is typically formed at an angle a in the range of 10° to 80° from the axis A, more preferably in the range of 20° to 70° and even more preferably in the range of 30° to 60°. Most preferably, the angle α is 52° from the axis A.

In the above-described embodiments, the circumferential wall 66, 166 has been planar, but it can instead take a bulbous, convex form 266, 566, as shown for example in the embodiment of the components 260, 560 of FIGS. 7 and 10. It can instead take a concave form. One particular example is shown in the embodiment of the component 360 of FIG. 8, where the concave surface of the circumferential wall 366 is substantially parallel to the convex external head surface 362, thereby defining a skirt 367 of substantially equal thickness depending from the periphery of the ceiling 368. FIG. 9 illustrates an alternative concave circumferential wall 466, which is not parallel to the outer surface but rather results in a skirt 467 of tapering thickness, being thinner towards the lower edge 463.

The ceiling 68, 168 is typically planar and formed substantially perpendicular to the axis A. However, in certain embodiments, such as illustrated in the embodiments of FIGS. 9 and 10, the ceiling 468, 568 is convex. A convex ceiling 468, 568 and/or a convex circumferential side wall 266, 566 can result in better load transfer to the surrounding bone.

In an alternative embodiment, shown in FIG. 11 by way of example, the internal cavity 664 is vaulted, defined by a chamfered circumferential wall 666 with a concave surface that meets at the axis A. In this embodiment, the ceiling can either be considered as having a vaulted concave surface that is contiguous with the surrounding circumferential wall 666.

In each of the illustrated embodiments, the pegs 70, 170, 470 extend from the circumferential wall 166, 466 and they each protrude beyond the plane P defined by the edge 63 of the external head surface 62. However, the distance beyond the plane P by which the pegs protrude is kept quite short so that the component has a relatively low profile in comparison to the prior art.

The length of fixation pegs, defined from the ceiling 68 to the free end 76 can be in the range of 20-150% of the height of the component head. In preferred embodiments, the pegs 70 protrude beyond the plane P by a distance that is 50% of the height of the component head (i.e. the distance along the axis A from the apex of the external head surface 62 to the plane P), which would typically be 8 mm.

So far, the invention has been described by reference to the component being secured to the underlying bone 14 by a plurality of fixation pegs that are arranged so as to be received in the peripheral portion 17b of an epiphyseal plate or line 17 of the bone.

However, alternative forms of fixation element are also envisaged, each sharing the characteristic that they too are arranged so as at least a part thereof is received in that peripheral portion 17b of an epiphyseal plate or line 17 of the bone.

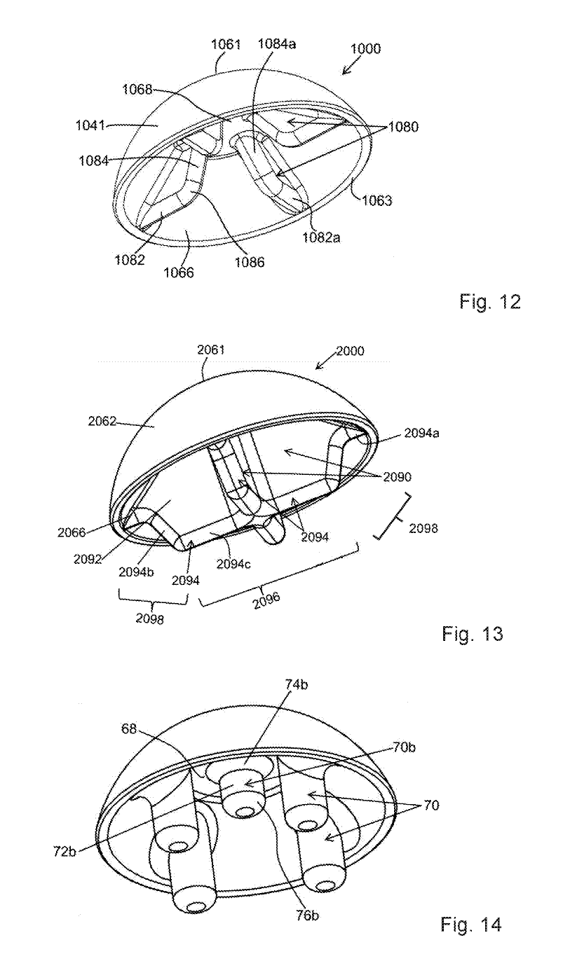

One example embodiment of this type of component 1000 is shown in FIG. 12, in which the fixation elements comprise fins 1080. Each fin 1080 comprises a first edge 1082 extending from a point on the circumferential wall 1066, and a second edge 1084 extending from a point on the ceiling 1068 to a point 1086 meeting the first edge. In this embodiment, the first edge 1082 is straight and extends substantially perpendicularly to the axis A. Since the point on the circumferential wall 1066 from which the first edge extends is close to the rim 1063, the first edge 1082 therefore extends substantially in line with the plane P. It should be understood that the first edge 1082 may instead be angled so that part of the fin 1080 projects beyond the plane P, or could be angled the opposite way so as to meet with the second edge at a more obtuse angle.

In embodiments in which the first edge 1082 does not project beyond the plane P, the component 1000 can have a particularly low profile (small height from the top 1061 of the hemi-spherical external bearing surface 1041 to the bottom of the fixation elements—i.e. the first edge 1082).

The second edge 1084 extends at a more acute angle to the axis A than the circumferential wall 1066, resulting in a fin 1080 that projects further from the circumferential wall 1066 towards the rim 1063 than it does closer to the ceiling 1068. If the second edge 1084 extended at the same angle as the circumferential wall 1066, then the fin would project an equal distance from the circumferential wall from the ceiling 1068 to the first edge 1082.

As illustrated, the junction 1086 between the first and second edges 1082, 1084 is rounded. In other embodiments, the junction could instead be sharp. Examples of such sharp junctions are shown in the embodiments of FIGS. 18a and 18b.

In FIG. 18a, each fin 1180 has a short, straight first edge 1182 extending substantially perpendicularly to the axis A, substantially aligned with the plane P. The second edge 1184 is curved, substantially parallel to the surface of the circumferential wall 1166. The first and second edges 1182, 1184 meet at a sharp junction 1186. In FIG. 18b, by contrast, each fin 1280 has a relatively long, inwardly curved first edge 1282. The second edge 1284 is relatively short and straight, extending at an angle substantially parallel to the axis A. The first and second edges 1282, 1284 meet at a sharp junction 1286. The skilled person will appreciate that many different combinations of such features can be employed to result in differently-configured fins. In particular, the angles of the first and second edges relative to their respective points of contact on the circumferential wall and on the ceiling, and relative to one another, as well as whether the edges are straight or curved, can be varied. In certain embodiments, the first and second edges could be contiguous with one another, rather than having a defined junction.

In FIG. 18c, each fin 1380 has a relatively long, outwardly concavely curved first edge 1382, which runs from a point on the circumferential wall 1366 quite close to the ceiling 1368 to the axis A. The second edge 1384 of each fin is aligned with the axis A and the fins 1380 are thus all joined at the axis. The embodiment of FIG. 18d is similar, but here the first edge 1482 of each fin 1480 is convexly curved.

Whatever combination of angles and curvature of the first and second edges, the first and second edges may either or both have a rounded profile 1082a, 1084a (as shown in FIG. 12), or have a flat profile 1282a, 1284a (as shown in FIG. 18b for example).

As with the pegs 70, the fins 1080 may be arranged symmetrically and equidistantly from one another, or could be arranged so as to maximise the prospects of being located in optimally dense bone, for example, their location could be optimised using on the results of the above outlined study. There may likewise be greater than or fewer than four fins. The embodiments of FIGS. 18a to 18d, for example, each include five fins.

Another alternative form of fixation element is the keel. One example embodiment of this type of component 2000 is shown in FIG. 13. Each keel 2090 extends across the whole of the internal cavity 2064, from a point on the circumferential wall 2066 to a corresponding point diametrically opposite. Each keel 2090 comprises a flat portion 2092 aligned parallel to and intersecting the axis A, the flat portion having a free edge 2094 opposite to the ceiling 2068.

In this embodiment, the free edge 2094 is symmetrical in the axis A and comprises a first portion 2094a that runs from a point on the circumferential wall 2066 inwardly a short way substantially parallel to and aligned with the plane P, a third portion 2094c that runs across a central part of the component 2000 parallel to the plane P, but further away from the top 2061 of the external bearing surface 2062 than the plane P, and a second portion 2094b that joins the first and third portions 2094a, 2094c. As such, a central part 2096 of each keel 2090 projects beyond the plane P, but only a relatively short distance to keep the height of the component as low as possible whilst still providing sufficiently secure attachment of the component 2000 to the underlying bone 14 by virtue at least of the provision of the peripheral parts 2098 of each keel 2090.

In this embodiment, a pair of such keels 2090 is provided, intersecting one another at right angles to form a cruciform fixation element. However, it will be appreciated that a greater number of keels could be provided, and that they may be arranged at any orientation relative to one another, depending on the number of keels and preferred locations for them.

The free edge 2094 of this embodiment has a rounded profile, but it could instead be flat.

In one sense, the keels can be thought of as special cases of the fins, where the second edges all meet at a central point aligned with the axis A.

Whatever form of fixation element is provided in the periphery of the component to be received in the corresponding peripheral region of the underlying bone (i.e. typically depending from the circumferential wall), one or more additional fixation elements may be provided to enhance the security of attachment.

FIGS. 14 to 17 illustrate some examples. In FIG. 14, the component corresponds to that shown and described with reference to FIGS. 4a and 4b in particular (so the same reference signs are used), but with the addition of a single supplementary fixation peg 70b projecting down centrally from the ceiling 68. The supplementary peg 70b comprises a substantially cylindrical body portion 72b and a wider base portion 74b, where the peg 70b is connected to the ceiling 68. The supplementary peg 70b has a rounded free end 76b. The supplementary peg 70b is typically shorter than the corresponding pegs 70, and would typically terminate between the ceiling 68 and the rim 63.

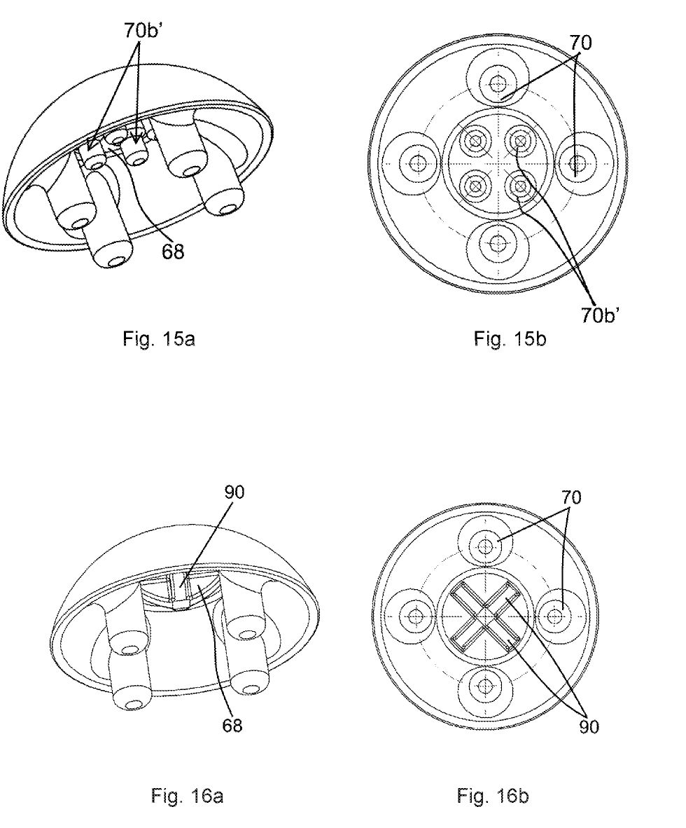

In the embodiment of FIGS. 15a and 15b, instead of a single central supplementary peg 70b, four such supplementary pegs 70b′ are provided extending from the ceiling 68. As best seen in FIG. 15b, the supplementary pegs 70b′ are arranged equidistantly and at 90° separation from one another, although off-set at 45° with respect to the corresponding primary fixation pegs 70.

In the embodiment of FIGS. 16a and 16b, the four supplementary pegs 70b′ are replaced instead by a pair of mini keels 90 extending from the ceiling 68, likewise off-set with respect to the corresponding primary fixation pegs 70 by 45°.

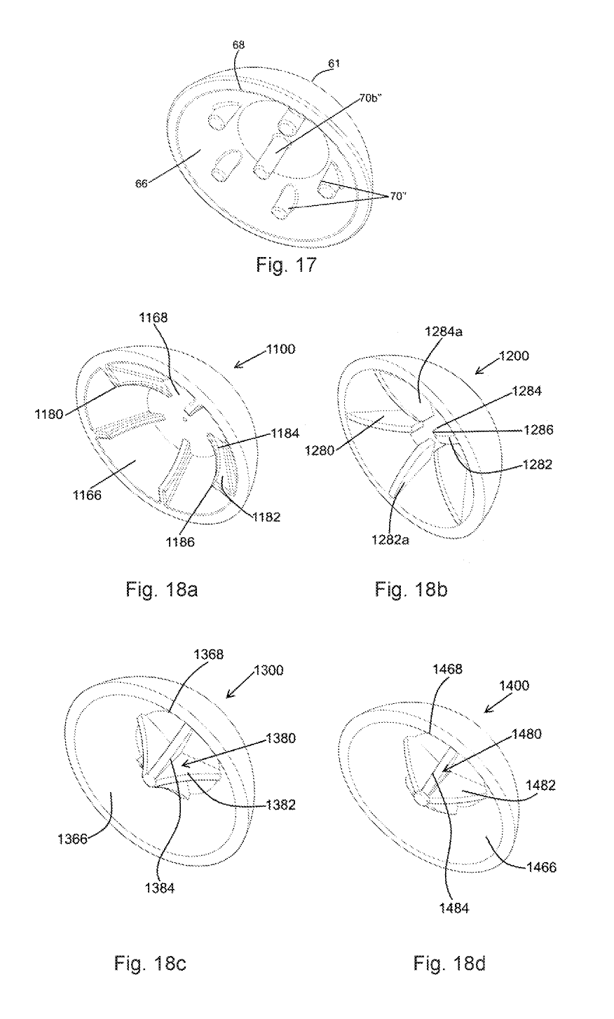

By way of one further example, FIG. 17 illustrates an embodiment in which there are five relatively short primary fixation pegs 70″, each extending from the circumferential wall 66 but not projecting beyond the plane P, in combination with a relatively long supplementary peg 70b″ projecting down centrally from the ceiling 68, the free ends 76″ of the primary fixation pegs 70″ and the free end 76b″ of the supplementary peg 70b″ all terminating at about the same distance from the top of the external bearing surface.

These are just some examples of suitable arrangements of primary fixation elements and supplementary fixation elements. As will be appreciated, the skilled person will be able to envisage numerous suitable combinations of the above-described features to result in a large variety of different fixation element configurations meeting the objectives of the invention.

Whichever embodiment is employed, the component 60 is formed from a single piece of material. The manufacturing process may depend on the material and on the configuration of the fixation elements in particular, but suitable manufacturing techniques include machining the component from a single block of raw material, or additive manufacture, which is particularly suited to patient-specific implementations.

The component is by preference manufactured from ceramic, due to its superior wear characteristics. However, any suitable medically-approved material may be employed. Examples of suitable materials therefore include: ceramic, stainless steel, cobalt chromium, titanium, Silicon nitride, carbon-reinforced PEEK, pyrocarbon, and suitable alloys thereof.

In certain embodiments, the component may have an osseo-conductive coating on the internal cavity 64—i.e. on either or both of the circumferential wall 66 and the ceiling 68. There may also or instead be such an osseo-conductive coating on the plurality of fixation elements. In addition or instead, the external bearing surface 62 may include a lubricious coating, as known in the art.

The subchondral humeral bone is stronger at the periphery of the epiphyseal plate (or line) than centrally and accordingly by providing fixation elements that extend into the peripheral portion 17b a stronger, more reliable attachment between the humeral component and the subchondral bone can be made, reducing the possibility of the above-described sink-hole effects. Moreover, by moving the fixation points towards the periphery, problems associated with induced rocking motions due to central fixation can be avoided.

The shape of the component may be determined pre-operatively, through known techniques including the use of computer-assisted image methods based on three-dimensional images of the patient's anatomy reconstructed from MRI, CT, ultrasound, X-ray, or other three- or two-dimensional medical scans of the patient's anatomy.

Such a component 10 may conveniently be manufactured using additive manufacturing techniques.

Rather than being made to be fully patient-specific, a more generic-shaped component blank can be provided, with only certain features tailored to suit a particular patient's requirements. For example, a generic blank can be provided, with only the fixation elements being located according to the patient's particular bone structure, as described above. This may be advantageous to reduce the pre-operative planning phase and to streamline the design and manufacture steps.

A range of different sizes of blanks can be provided to accommodate different-sized patients; for example, small, medium and large components. With a greater number of options, a greater sized inventory of blanks must be maintained, so it is preferable to keep the number of options to a minimum reasonable whilst still providing adequate fit to the patient.

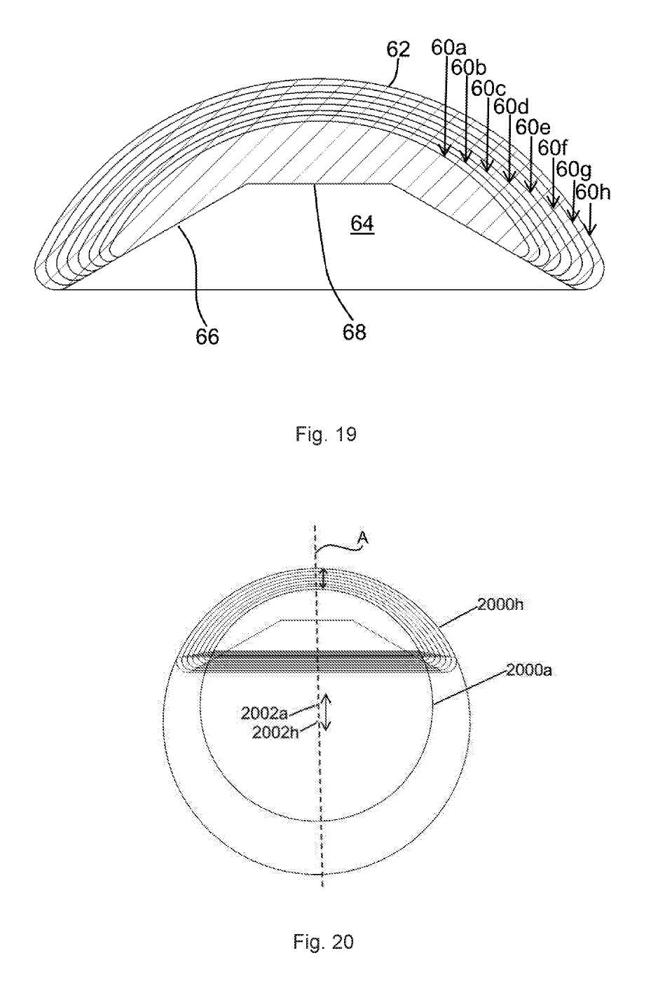

FIGS. 19 and 20 depict a range of different sized components 60a-60h, overlaid on one another to illustrate their differing external profiles and their common internal profile. Each of the differently-sized components 60a-60h has an external bearing surface 62 with a radius of curvature ranging from a smallest size (for the component 60a) to a largest size (for the component 60h). The respective arcs 2000a-2000h described by the radii are centred on a common axis A for all of the components. According to one embodiment, the centres of rotation of the arcs 2002a-2002h are offset from one another along the axis A (in a medial direction), as shown in FIG. 20. This lateralisation of the radii of curvature of the respective external bearing surfaces 62 can help to avoid joint overstuffing.

The internal profiles of the respective differently-sized components 60a-60h (i.e. the shape of the internal cavity 64 as defined by the chamfered circumferential wall 66 and the ceiling 68) are common.

To fit the component 60 in position on the patient's humeral head 14, a surgeon would prepare the surface of the patient's humeral head, resecting a portion thereof, if appropriate, so as to be sized and shaped to match the shape of the internal cavity 64 of the pre-operatively determined component 60. The resected volume would thus be sized and shaped such that the component 60 will, once fitted in place, be in the correct position with respect to the patient's glenoid cavity surface (or glenoid prosthesis, if that is also being replaced), the external bearing surface 62 thus being in registration with the glenoid cavity for articulation therewith and the component being substantially contiguous with the topology of the surrounding bone 14.

The surgeon would then prepare the bone surface by drilling or otherwise forming holes at the pre-operatively determined anchor locations (not shown), sited to receive the fixation elements. These bone preparation steps are preferably carried out through one or more minimally-invasive incisions.

If, as described above, a range of differently-sized components 60 is available, each having a common internal profile, then the surgeon is afforded flexibility to change the size of component 60 intraoperatively, even after the resection step. Typically, a trial component without pegs 72 or other fixation features may first be fitted in place to determine if it is the correct size for the patient. Once correct sizing is confirmed, then the bone surface preparation steps would be carried out, specific to the locations of the fixation features for the selected size of component 60.

The term ‘minimally invasive surgical procedure’ is considered to have an established meaning within the art and is intended to encompass, inter alia, arthroscopic and mini-open surgical approaches, including those using multiple minimally-invasive incisions.

The component 60 can then be inserted, typically through one of the minimally-invasive incisions, to be fitted in place. The fixation elements are received in the holes with an interference fit for cementless attachment of the component 60 to the bone.

Because the component 60 has a low profile, the surgical procedure can advantageously be carried out via posterior access. Moreover, even if the procedure is carried out anteriorly, the chamfered shape of the internal cavity 64 provides good access therepast to the glenoid. The Chamfer shape also preserves the stronger bone above the anatomical neck as shown by the investigators' study mentioned above.

As well as the above-described advantages resulting from the location of the fixation elements within the peripheral region of the humeral bone 14, an additional benefit is that this configuration leaves the central region of the bone undamaged, meaning that it remains available for use in a revision procedure, such as implanting a conventional, cemented component with a central stem in place.

It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail. Accordingly, individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure as defined by the accompanying claims.

1. A prosthetic humeral head component comprising:
a convex external head surface;
an internal cavity formed opposite to the head surface; and
a plurality of fixation elements protruding from said cavity oppositely to said head surface for location into a peripheral portion of an epiphyseal plate of a humerus to secure the component to the humerus, wherein the component is formed as a single, unitary piece.

2. The component of claim 1, wherein the component is rotationally symmetrical about an axis.

3. The component of claim 2, wherein the internal cavity is substantially frusto-conical, defined by a chamfered circumferential wall and a ceiling.

4-6. (canceled)

7. The component of claim 3, wherein the circumferential wall has a concave surface, wherein the concave surface of the circumferential wall is substantially parallel to the convex external head surface, thereby defining a skirt of substantially equal thickness depending from the periphery of the ceiling.

8. (canceled)

9. The component of claim 2, wherein the internal cavity is vaulted, defined by a chamfered circumferential wall with a concave surface that meets at the axis.

10. The component of claim 3, wherein the circumferential wall has a convex surface.

11. The component of claim 3, wherein the fixation elements extend from said circumferential wall.

12. (canceled)

13. The component of claim 1, wherein the fixation elements comprise pegs.

14-18. (canceled)

19. The component of claim 1, wherein the fixation elements comprise fins.

20. The component of claim 3, wherein the fixation elements comprise fins, and wherein each fin comprises a first edge extending from a point on the circumferential wall, and a second edge extending from a point on the ceiling to a point meeting the first edge.

21. The component of claim 20, wherein the first edge extends substantially perpendicularly to the axis.

22. The component of claim 20, wherein at least one of the first edge and the second edge is curved.

23. (canceled)

24. The component of claim 20, wherein the second edge extends at a more acute angle to the axis than the circumferential wall.

25. The component of claim 20, wherein at least one of:
the first and second edges each have a rounded profile,
the junction between the first and second edges is rounded; and
the first and second edges are contiguous with one another.

26-29. (canceled)

30. The component of claim 1, wherein the fixation elements comprise keels, each keel extending across the whole of the internal cavity.

31. The component of claim 3, wherein the fixation elements comprise keels, each keel extending across the whole of the internal cavity, and wherein each keel comprises a flat portion aligned parallel to and intersecting the axis, the flat portion having a free edge opposite to the ceiling.

32-35. (canceled)

36. The component of claim 1, wherein the component is formed from a single piece of material, selected from: a ceramic or a metallic alloy, such as cobalt-chrome or titanium.

37. The component of claim 1, further comprising an osseo-conductive coating on at least one of the internal cavity and the plurality of fixation elements.

38. The component of claim 1, wherein each of the plurality of fixation elements is positioned such that its centre is ata radial distance from a central axis of the component through the convex external head surface that is at least 70% of the radius of the component from the central axis.

39. The component of claim 1, wherein each of the plurality of fixation elements is positioned such that its centre is at a radial distance of at least 15 mm from a central axis of the component through the convex external head surface.
